(19) United States
(12) Patent Application Publication
(10) Pub. No.: US 2010/0302501 A1
(43) Pub. Date: Dec. 2, 2010

(54) EYE AND EAR PROTECTION DEVICE

(76) Inventor: Greggory R. Hansen, Lafayette, CA (US)

Correspondence Address: Bay Area Technology Law Group PC, Suite 404, 500 Sansome Street, San Francisco, CA, 94111, US

(21) Appl. No.: 12/476,432

(22) Filed: Jun. 2, 2009

(51) Int. Cl. G02C 11/00; A41D 13/05; A61F 9/00

(52) U.S. Cl. 351/158; 2/423; 2/15

(57) ABSTRACT

A device for facilitating eye and ear protection. The device includes an eyeglass frame having left and right temple pieces, a lens support member and lenses appended to the lens support member. Left and right ear plug housings are supported by each of the left and right temple pieces and ear plugs being removably retained within the ear plug housings. Umbilicals connect the ear plugs to the eyeglass frame which are of a length to enable a user to remove the ear plugs from their housings and to insert the ear plugs within ear canals of the user whenever the eyeglass frame is worn.

### TECHNICAL FIELD

The present invention involves a device which is simple and convenient to use and capable of protecting a user's eyes and ears whenever eye and ear protection is deemed advisable. When only eye protection is required, the ear protection component of this invention can be stored unobtrusively thus not interfering with the usefulness of the invention as a whole.

### BACKGROUND OF THE INVENTION

It is quite common in industrial as well as in leisure settings to provide one with both eye and ear protection. Industrially, flying debris accompanied by high decibel processing noise oftentimes dictate the need for both eye and ear protection. Many industries actually mandate that eye and ear protection be made available to anyone working in a particular locale.

Even when industrial safety requirements do not mandate the use of eye and ear protection, many activities would benefit if both forms of protection were made readily available. For example, if one was to attend an automobile race, and particularly an event involving dragsters, ear protection is almost always recommended. Exposure to high SPLs for extended periods of time can cause irreversible hearing loss. Many of such events are also carried out in mid day during hot summer months and those attending such events routinely use sunglasses as a form of eye protection.

Whether the setting is industrial or recreational, availability of ear protection in conjunction with eye protection is not always available Most commonly, one might carry safety or sunglasses but ear protection, generally in the form of ear plugs, quite often, are lost as the size of such objects makes losing them a constant occurrence. Thus, when one needs ear protection, ear plugs can simply not be found.

It is thus an object of the present invention to provide a device in which ear protection is retained with eye protection for selective use when needed.

It is a further object of the present invention to selectively provide ear protection which, when not needed, is capable of withdrawing within an eyeglass frame to ensure that the ear protection is always available and yet unobtrusive when not needed.

These and further objects will be more readily appreciated when considering the following disclosure and appended claims.

### SUMMARY OF THE INVENTION

A device for facilitating eye and ear protection. The device includes an eyeglass frame having left and right temple pieces, a lens support member and lenses appended to the lens support member. Left and right ear plug housings are supported by each of the left and right temple pieces and ear plugs being removably retained within the ear plug housings. Umbilicals connect the ear plugs to the eyeglass frame which are of a length to enable a user to remove the ear plugs from their housings and to insert the ear plugs within ear canals of the user whenever the eyeglass frame is worn.

### BRIEF DESCRIPTION OF THE FIGURES

FIG. 1 is a perspective view of the device of the present invention.

FIG. 2 is a cut away view of an umbilical reel take up device used as a preferred embodiment

### DETAILED DESCRIPTION OF THE INVENTION

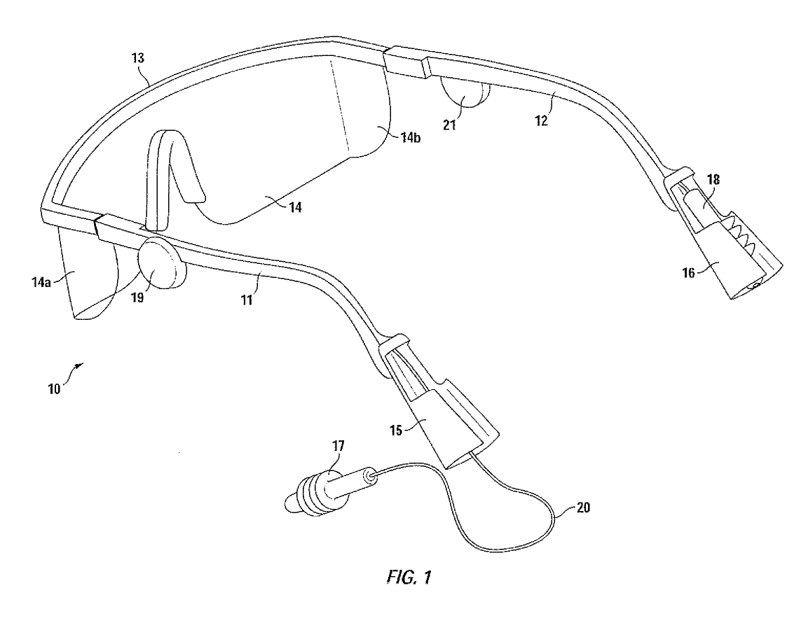

As depicted in FIG. 1, device 10 comprises an eyeglass frame having left and right temple pieces, 11 and 12 and lens support member 13. Appended to lens support member 13 are lenses 14, this example, being safety lenses having wrap around portions 14a and 14b, typical of safety glasses. However, as noted previously, the present invention can be used with recreational eyewear, such as sunglasses which would be devoid of segments 14a and 14b.

The present invention also includes left and right housings 15 and 16 supported by left and right temple pieces 11 and 12, respectively. Although such housings can be of any shape to facilitate their selective retention of ear plugs, it is convenient to fabricate them as shown being frustum shaped and ideally sized for the frictional retention of ear plugs 17 and 18, respectively thereon.

FIG. 1 shows left ear plug 17 removed from housing 15 while right ear plug 18 remains supported within housing 16. In either case, the ear plugs are connected to umbilicals, umbilical 20 being shown as connected to left ear plug 17 while its corresponding umbilical cannot be seen as it is channeled within right temple piece 12. Although the umbilicals can be of any particular length, they are ideally of a length at least sufficient to enable a user upon removal of ear plugs 17 and 18 from housings 15 and 16 to insert the ear plugs within ear canals of the user when the eyeglass frame is worn

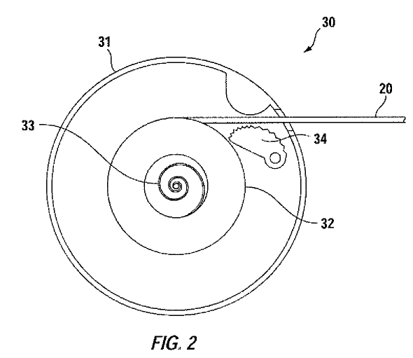

Broadly, the umbilicals appended to each ear plug can simply hang from the eyeglass frame whether ear plug 17 and 18 are in use or stored within their respective housings. These umbilicals can be made of any type of string, thread or cord and can be made to be elasticized if desired. However, in order to maintain a neat and well tailored appearance, as a preferred embodiment, left and right reel elements 19 and 21 can be provided which, in the example shown in FIG. 1, are fixedly appended to temple pieces 11 and 12. In this preferred embodiment, the umbilicals pass from their respective ear plugs through housings 15 and 16 and are channeled within temple pieces 11 and 12 for pick up by reel elements 19 and 21. In this regard, reference is made to FIG. 2.

Turning to FIG. 2, a reel element is shown. Specifically, reel element 30 is provided with housing 31 and, retained therein, is spool 32. Spring 33 biases spool 32 to receive umbilical 20. Thus, as umbilical 20 is withdrawn from housing 31, spring 33 is tensioned providing resistance to the payout of umbilical 20. However, the reel mechanism includes ratchet 34, patterned after a similar mechanism used in common window shades. This works as a locking assembly for paying out the umbilicals until the umbilicals are urged away from the spool to engage the rewinding mechanism to cause umbilical 20 to wrap about spool 32. Thus, when not needed, left ear plug 17 will remain within housing 15 just as ear plug 18 remains within housing 16 as shown. Excess umbilical which would ordinarily drape from this device when the ear plugs are not in use, are taken up within reels 19 and 21.

Thus, device 10 can be used simply as protective or decorative eyewear until ear protection is necessary. In that event, a user simply employs one's thumb and forefinger to frictionally remove ear plugs 17 and 18 from housings 15 and 16 to thereupon fit the ear plugs within his or her ear canals No longer need one concern oneself with the loss of ear plugs or in not being prepared for ear protection whenever needed.

What is claimed is:

1. A device for facilitating eye and ear protection comprising an eyeglass frame having left and right temple pieces, a lens support member and lenses appended to said lens support member, left and right ear plug housings supported by said left and right temple pieces and left and right ear plugs removably retained within said left and right ear plug housings and umbilicals connecting said ear plugs to said eyeglass frame of a length to enable a user to remove said ear plugs from said ear plug housings and to insert said ear plugs within air canals of said user when said eyeglass frame is worn.

2. The device of claim 1 wherein said ear plugs are sized to frictionally fit within said ear plug housings preventing their inadvertent removal from said housings.

3. The device of claim 1 wherein said ear plug housings are located proximate ends of said temple pieces.

4. The device of claim 1 further comprising left and right reel elements each having a spool for receiving said umbilicals when said ear plugs are within said ear plug housings and for paying out said umbilicals when said ear plugs are withdrawn from said ear plug housings

5. The device of claim 4 wherein each of said reel elements comprises a spring loaded locking assembly for retaining the pay out of said umbilicals until said umbilicals are urged away from said spool to engage rewinding of said umbilicals thereon.

6. The device of claim 1 wherein said eye protection comprises safety glasses

7. The device of claim 1 wherein said eye protection comprises sunglasses.
